(19) United States
(12) Patent Application Publication
(10) Pub. No.: US 2007/0143147 A1
(43) Pub. Date: Jun. 21, 2007

(54) Systems, methods and apparatus of an image network adapted to process and display image data from a mobile digital imaging system

(75) Inventors: Scott William Petrick, Sussex, WI (US); Nirmal Mukund Kari, Waukesha, WI (US)

Correspondence Address: RAMIREZ & SMITH, PO BOX 341179, AUSTIN, TX, 78734, US

(73) Assignee: General Electric Company, Schenectady, NY (US)

(21) Appl. No.: 11/293,532

(22) Filed: Dec. 3, 2005

(51) Int. Cl. G06F 19/00; A61B 5/00

(52) U.S. Cl. 705/3

(57) ABSTRACT

Systems, methods and apparatus are provided through which in some embodiments, a mobile digital electronic imaging system includes an adapter operable to transmit a digital image to a network. In some embodiments, a network is operable to receive a digital image from a mobile digital electronic imaging system and perform functions upon the digital image, such as display the image or perform processing on the image.

### FIELD OF THE INVENTION

This invention relates generally to diagnostic medical imaging systems, and more particularly to display of images from mobile imaging systems.

### BACKGROUND OF THE INVENTION

Conventional mobile medical diagnostic-imaging systems, such as mobile digital X-Ray imaging systems, are in widespread use by hospitals, trauma centers, and clinics. Mobile digital X-Ray imaging systems consist primarily of an X-Ray generator and an X-Ray tube mounted on a motorized chassis powered from a battery. The imaging is performed on imaging media of either chemical film or an electronic detector.

Mobile medical X-Ray imaging systems are often used when a patient is unable to move to a fixed-based X-Ray imaging system. To image the patient, a clinician moves the mobile medical X-Ray imaging system to the patient, positions the tube on one side of the patient, places either a film screen cassette or an electronic detector on the other side of the patient, and images an exposure. For chemical film imaging, the clinician walks the film cassette to a film processor, develops the film and finally slips the finished film on a light box to make sure that the exposure was of diagnostic quality, considering, among other things, exposure technique and patient positioning. For electronic detectors, the electronic image data is stored on electronic media and physically transported to an electronic system that is capable of processing and/or displaying the image.

However, the process of viewing the image from either electronic image detectors or chemical film cassettes is laborious, thus expensive, in an electronic era. More specifically, imaging on film cassette or electronic detectors both require physical transportation of the imaging media to another location. Medical facilities would benefit from the cost savings that could result from reducing or eliminating the need to physically transport the imaging media.

More recent conventional mobile medical digital X-Ray imaging systems are manufactured with a flat panel display. However, the flat panel of the display mobile medical X-Ray imaging systems increases the size and weight of the mobile medical X-Ray imaging system, reducing the mobility of the mobile medical X-Ray imaging system. The display also increases the power drain on the battery, which reduces the operable time frame of the mobile medical digital X-Ray imaging system.

More recent conventional mobile medical digital X-Ray imaging systems are manufactured with a flat panel display. A display incorporated into the mobile medical digital X-Ray imaging system facilitates immediate display of the image, which in turn provides immediate confirmation to the clinician after imaging a patient that that appropriate exposure and positioning techniques had been used. Also, in emergency situations, an immediate diagnosis can be initiated from the displayed image. However, the display adds to the cost of the mobile medical digital X-Ray imaging system. In addition, mobile medical digital X-Ray imaging systems that are manufactured with a flat panel display also have increased size, weight and power consumption that results in reduced mobility and reduced battery life of the mobile medical digital X-Ray imaging system.

For the reasons stated above, and for other reasons stated below which will become apparent to those skilled in the art upon reading and understanding the present specification, there is a need in the art to process and/or display an image from a mobile digital medical diagnostic imaging system that does not significantly increase the cost, weight, size and power consumption of the mobile digital medical diagnostic imaging system.

### BRIEF DESCRIPTION OF THE INVENTION

The above-mentioned shortcomings, disadvantages and problems are addressed herein, which will be understood by reading and studying the following specification.

Such a workflow makes more use of the efficiencies afforded by the computer technology that includes the portable flat panel solid state X-Ray detector.

In one aspect, existing mobile digital X-Ray imaging systems are retrofitted with flat panel liquid-crystal displays (LCDs).

In another aspect, a mobile digital electronic imaging system includes a mobile X-Ray unit base, a column operably coupled to the mobile X-Ray unit base, a horizontal arm operably coupled to the column, an X-Ray source operably coupled to the horizontal arm, a processor operably coupled to the X-Ray source, and one or more network adapters mounted on the mobile X-Ray unit base and operably coupled to the processor.

In yet another aspect, each of the plurality of network adapters of the mobile digital X-Ray imaging system further comprise an Ethernet-compliant network adaptor.

In still another aspect, a system to process a digital image from a mobile digital medical diagnostic imaging system, includes a network, image processing system operably coupled to the network, and a network server operably coupled to the network, the network server operable to receive a digital image from the mobile digital medical diagnostic imaging system through the network and operable to transmit the digital image to the image processing system.

In a further aspect, a method to manage images between a mobile digital medical diagnostic imaging system and a network includes receiving from the mobile digital medical diagnostic imaging system, a digital image and request in reference to the digital image, identifying a resource on the network to perform the request on the digital image and transmitting the request and the digital image to the identified resource.

In yet a further aspect, a directory provides an association between an identity of each resource on the network, the physical location of the resources on the network and an availability of the resource on the network.

Apparatus, systems, and methods of varying scope are described herein. In addition to the aspects and advantages described in this summary, further aspects and advantages will become apparent by reference to the drawings and by reading the detailed description that follows.

### BRIEF DESCRIPTION OF THE DRAWINGS

FIG. 1 is a block diagram that provides an overview of a system to capture a digital image at a mobile medical digital X-Ray imaging system and to display the digital image;

FIG. 2 is a side view of mobile digital X-Ray imaging system according to an embodiment having one or more network interfaces;

FIG. 3 is a diagram of apparatus according to an embodiment that captures a digital image at a mobile medical digital X-Ray imaging system and displays the digital image;

FIG. 4 is a flowchart of a method according to an embodiment to manage digital images from a mobile digital medical diagnostic imaging system on a network;

FIG. 5 is a flowchart of a method according to an embodiment to identify a resource on a network to perform a request on a digital image;

FIG. 6 is a block diagram of the hardware and operating environment in which different embodiments can be practiced;

FIG. 7 is a block diagram of the mobile digital medical diagnostic imaging system processor hardware and operating environment to manage digital images;

FIG. 8 is a block diagram of a network server hardware and operating environment to manage digital images from a mobile digital medical diagnostic imaging system; and

FIG. 9 is a diagram of a layout of a table of a directory for reference in identifying one or more resource on the network to process requests.

### DETAILED DESCRIPTION OF THE INVENTION

In the following detailed description, reference is made to the accompanying drawings that form a part hereof, and in which is shown by way of illustration specific embodiments which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

The detailed description is divided into five sections. In the first section, a system level overview is described. In the second section, apparatus of embodiments are described. In the third section, embodiments of methods are described. In the fourth section, the hardware and the operating environment in conjunction with which embodiments may be practiced are described. Finally, in the fifth section, a conclusion of the detailed description is provided.

### System Level Overview

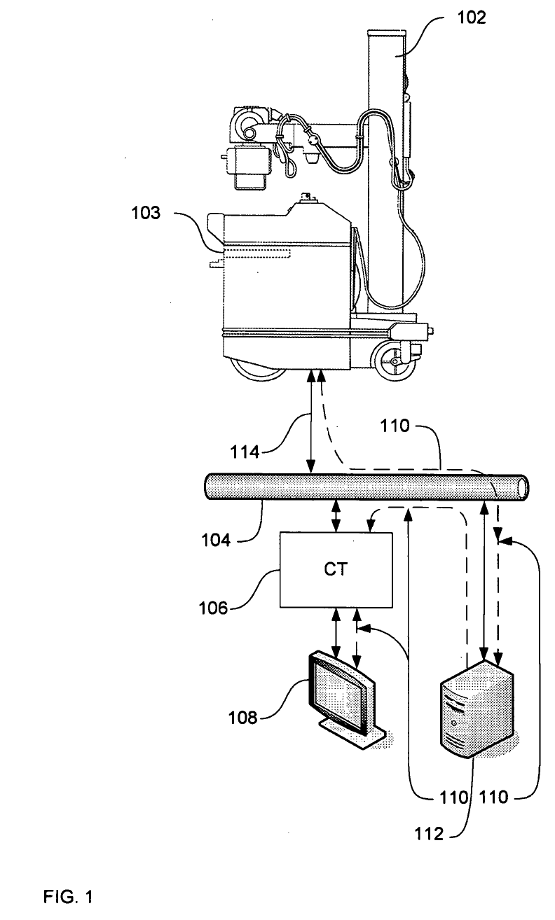

FIG. 1 is a block diagram that provides an overview of a system to capture a digital image at a mobile medical digital X-Ray imaging system and to process the digital image. System 100 solves the need in the art to display an image from a mobile medical digital X-Ray imaging system that does not significantly increase the cost, weight, size and power consumption of the mobile medical digital X-Ray imaging system.

System 100 includes a mobile digital medical diagnostic imaging system 102, such as the mobile X-Ray digital imaging system shown in FIG. 1. The mobile digital medical diagnostic imaging system 102 includes a digital X-Ray detector 103 and is operably coupled to a network 104, such as an Ethernet compliant network shown in FIG. 1. In some embodiments, the network is a network that is specifically adapted to function as a medical imaging network.

System 100 also includes an image processing system 106 that has a display device, such computed tomography (CT) image processing system that includes display device 108.

After the mobile digital medical diagnostic imaging system 102 captures a digital image, the digital image is transmitted to the network 104 along communication path 110. A server 112 on the network 104 receives the digital image and transmits the digital image along communication path 110 to an image processing system that has display device, such CT image processing system 106 that includes display device 108, whereupon the digital image is displayed on the display device 108. Thus, displaying an image that is captured on the mobile digital medical diagnostic imaging system 102 does not require a display device on the mobile digital medical diagnostic imaging system 102. Thus, system 100 does not require the expense, weight, additional size or power drain on the battery of the mobile digital medical diagnostic imaging system 102. These economies are achieved simply by operably coupling the mobile digital medical diagnostic imaging system 102 to the network, the network already having the apparatus that provides the capability to display the image.

System 100 provides a means of accessing and using the display and image processing capability of the network. Where the display and imaging capability of the network is significant, this is a capability of system 100. For example, when the network 104 has sophisticated abilities such as a network that provides interactive access to images encoded in accordance with the Digital Imaging and Communications in Medicine (DICOM) standard, or three-dimensional (3D) software tools to handle high exam volumes, large data sets and faster scan times, such as provided by the Advantage Imaging Network® or Advantage Workstation® manufactured by General Electric Healthcare of Waukesha, Wis. These capabilities are very expensive to provide in the mobile digital medical diagnostic imaging system 102. However, where such sophisticated abilities are available to the mobile digital medical diagnostic imaging system 102, it is considerably more economical to operably couple the mobile digital medical diagnostic imaging system 102 to the network, while also avoiding adding the weight, space requirements and power consumption requirements of the imaging apparatus that would provide that capability on the mobile digital medical diagnostic imaging system 102. System 100 avoids having to provide sophisticated and expensive imaging processing and display functions in redundant and duplicate quantities.

A coupling 114 between the mobile digital medical diagnostic imaging system 102 and the network can be accomplished in one of a number of means. For example, a wireless connection is implemented between the mobile digital medical diagnostic imaging system 102 and the network. In some embodiments, a wired connection is implemented. In some embodiments, and coupling 114 includes the Internet.

While the system 100 is not limited to any particular mobile digital medical diagnostic imaging system 102, network 104, image processing system 106, display device 108, communication path 110, server 112, or coupling 114, for sake of clarity a simplified mobile digital medical diagnostic imaging system 102, network 104, image processing system 106, display device 108, communication path 110, server 112, and coupling 114 are described.

### Apparatus Embodiments

In the previous section, a system level overview of the operation of an embodiment was described. In this section, the particular apparatus of such an embodiment are described by reference to a series of diagrams.

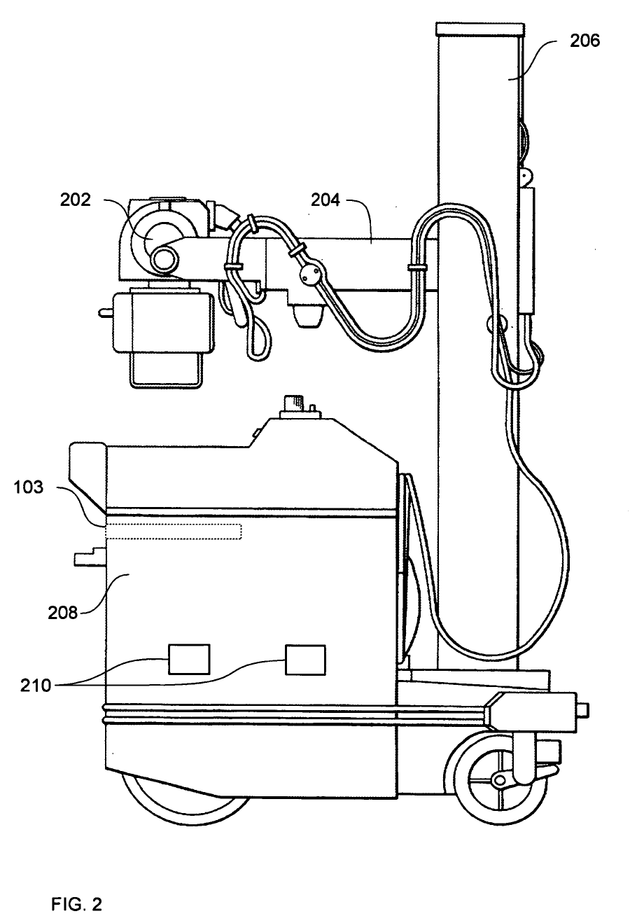

FIG. 2 is a side view of mobile digital X-Ray imaging system 200 according to an embodiment having one or more network interfaces. Apparatus 200 solves the need in the art to display an image from a mobile medical digital medical diagnostic imaging system that does not significantly increase the cost, weight, size and power consumption of the mobile medical digital medical diagnostic imaging system.

Mobile digital X-Ray imaging system 200 includes an X-Ray source 202 that is mounted to the end of a horizontal arm 204. The X-Ray source 202 is positionable over an area of concern on a patient. The X-Ray source 202 is typically mounted through a gimbal type arrangement in which a column 206 is required to rotate to move the X-Ray source from the park position on the mobile X-Ray unit base 208 to the appropriate position in order to take an X-Ray image of the patient.

Mobile digital X-Ray imaging system 200 also includes one or more network adapters 210. Two network adapters in the plurality of network adaptors 210 are shown in FIG. 2, but any number of network adapters can be implemented. In implementations where two or more network adapters 210 is include, one of the network adapters 210 is conventionally is used to connect to an external digital X-Ray detector. One of the other additional network adapters 210 is used as an interface to an electronic system that is operable to display an image from the mobile digital X-Ray imaging system 200, such as system 100. At least one of the network adapters 210 is a conventional network adapter, such as an Ethernet adapter. In some instances, the horizontal arm 204 is extended, and the digital X-Ray detector 103 is attached via a tether (not shown) to the mobile X-Ray unit base 208. The tether would contain both power connection and a network cable.

In some embodiments, both the mobile X-Ray unit base 208 and the digital X-Ray detector 103 have network adapters. In that case, both the mobile X-Ray unit base 208 and the digital X-Ray detector 103 operably couple to separate network jacks. In some embodiments of such a case, the mobile X-Ray unit base 208 includes only one network adapter.

The graphical depiction of the size of the network adapters 210 is increased relative to the mobile digital X-Ray imaging system 200 in FIG. 2 in order to improve legibility of the network adapters 210. The actual network adapters are usually smaller in size relative to the mobile digital X-Ray imaging system 200.

In an alternative embodiment, mobile digital X-Ray imaging system 200 includes only one network adapter to communicate to the electronic system that is operable to display an image from the mobile digital X-Ray imaging system 200, such as system 100.

In one example where of the network adapters 210 are Ethernet network adapters, the Ethernet network adapter provides a connection to a mobile digital medical diagnostic imaging system, such as the mobile digital medical diagnostic imaging system 102 of FIG. 1 through an Ethernet compliant communication channel. In some embodiments, the Ethernet compliant communication channel couples to a router, that in turn in couples to the Internet, that in turn is connected to a network, such as network 104 in FIG. 1 that provides display and processing apparatus and capability on a digital image that is captured by the mobile digital medical diagnostic imaging system 102. In some embodiments, the Ethernet compliant communication channel is a direct wired connection to the network, such as network 104 in FIG. 1. As an alternative to Ethernet, other conventional network protocols can be used, such as LocalTalk that was developed by Apple Computer, Inc., token ring protocol that was developed by IBM, fiber distributed data interface (FDDI), and asynchronous transfer mode (ATM). In addition, any conventional network topology can be used, such as linear bus, star, tree, star-wired ring or dual ring.

Mobile digital X-Ray imaging system 200 shows a network adaptor for a wired Ethernet connector. However mediums other than wires can be implemented, such as wireless connection (e.g. infrared or radio) to couple or connect the mobile digital X-Ray imaging system 200 to a network.

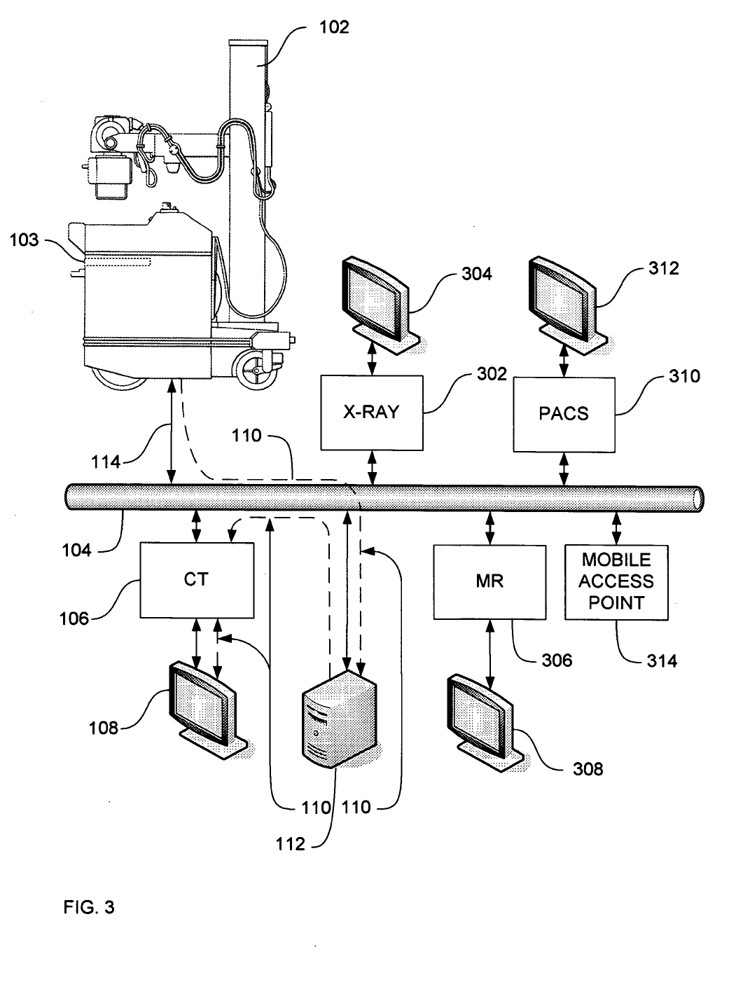

FIG. 3 is a diagram of apparatus 300 according to an embodiment that captures a digital image at a mobile medical digital X-Ray imaging system and displays the digital image. Apparatus 300 solves the need in the art to process and/or display an image from a mobile medical digital medical diagnostic imaging system that does not significantly increase the cost, weight, size and power consumption of the mobile medical digital medical diagnostic imaging system.

In addition to the elements of FIG. 1, apparatus 300 includes a number of additional image processing systems that provide processing and that have a display device, such as X-Ray image processing system 302 that includes display device 304 and magnetic resonance (MR) image processing system 306 that includes display device 308. In some embodiments, network 104 also includes a picture archive and communication system (PACS) 310 that includes a display 312. The PACS 310 is a computer network processing and storing digitized radiologic images and reports.

In some embodiments, the network 104 also includes a mobile access point 314. The mobile access point 314 provides an access point to the network 104 by a mobile digital medical diagnostic imaging system to the network 104. For example, mobile digital medical diagnostic imaging system 102 is operable to access the network 104 through the mobile access point 314.

Thus, the network 104 is operable to receive a digital image from the mobile digital medical diagnostic imaging system 102, and the mobile digital medical diagnostic imaging system 102 can avail itself of the processing and imaging capabilities of the network 104 and the image processing system 106, 302 306 and the PACS 310. In turn, apparatus 300 avoids having to redundantly and duplicatively provide sophisticated and expensive imaging processing and display finctions of the image processing system 106, 302 306 and the PACS 310 on the mobile digital medical diagnostic imaging system 102, which is considerably more economical. Apparatus 300 also avoids adding the weight and space requirements and power consumption requirements of the processing and display apparatus to the mobile digital medical diagnostic imaging system 102, which helps retain the mobility of the mobile digital medical diagnostic imaging system 102, while providing the processing and display finctions to the mobile digital medical diagnostic imaging system 102.

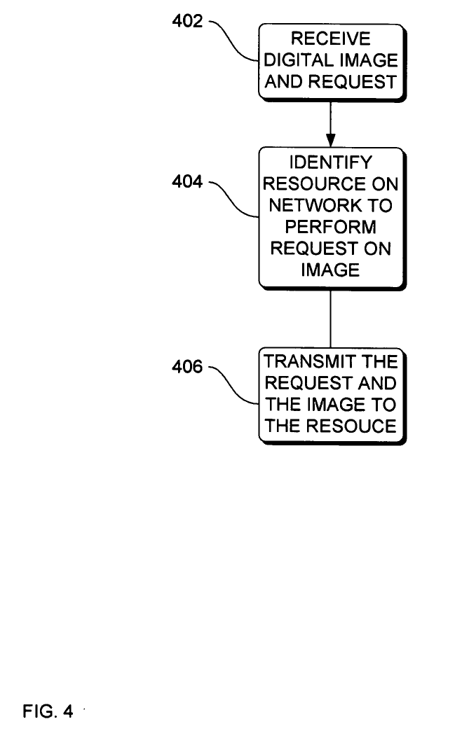

Method 400 in FIG. 4 below describes processes performed by server 112.

### METHOD EMBODIMENTS

In the previous section, apparatus of the operation of an embodiment was described. In this section, the particular methods performed by a network server, such as network server 112 in FIG. 1, of such an embodiment are described by reference to a series of flowcharts.

FIG. 4 is a flowchart of a method 400 according to an embodiment to manage digital images from a mobile digital medical diagnostic imaging system on a network. Method 400 includes receiving 402 a digital image and receiving a request in reference to the digital image. The digital image is received from a mobile digital medical diagnostic imaging system, such as mobile digital medical diagnostic imaging system 102 in FIG. 1 and FIG. 3 above, or mobile digital X-Ray imaging system 200 in FIG. 2 above. In some embodiments of the request, the request is a request is request to display the digital image, in some embodiments the request is a request to process the digital image, in some embodiments, the request is a request to process and display the digital image.

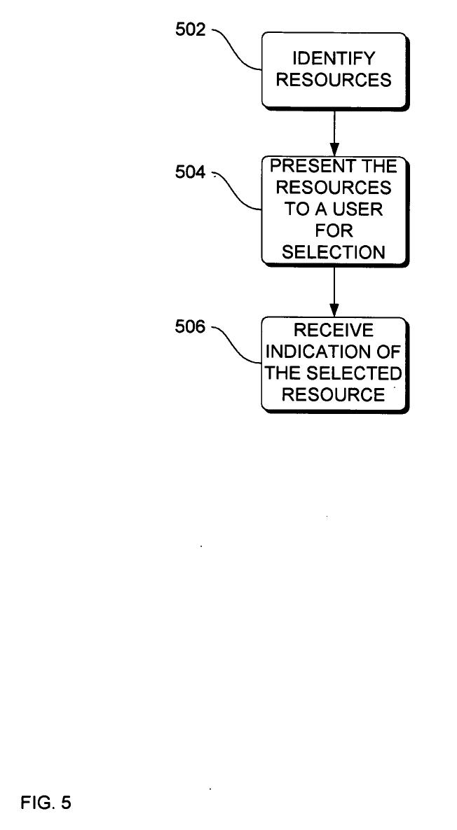

Method 400 also includes identifying 404 a resource on the network, such as network 102 in FIG. 1 or FIG. 3 above, to perform the request on the digital image. One embodiment of identifying 404 is described below in FIG. 5. In some embodiments, the network resource is an image processing system, such as image processing system 106 in FIG. 1 and FIG. 3. Some embodiments of the image processing system include an X-Ray image processing system, such as X-Ray image processing system 302 in FIG. 3, a CT image processing system such as CT image processing system 106 in FIG. 1 and FIG. 3 above, a MR image processing system, such as MR image processing system 306 in FIG. 3 above, or a picture archive and communication system such as PACS 310 in FIG. 3 above.

Some embodiments of identifying 404 include identifying a resource on the network to perform the request on the digital image in reference to a directory of a plurality of resources on the network. In some embodiments, the directory provides an association between an identity of each resource on the network, the physical location of the resources on the network and an availability of the resource on the network, as shown below in FIG. 10.

Subsequently, method 400 includes transmitting 406 the request to the identified resource and transmitting the digital image to the identified resource.

In some embodiments of method 400, before the request and digital image are received by the network, the network is polled for the request by the server.

FIG. 5 is a flowchart of a method 500 according to an embodiment to identify a resource on a network to perform a request on a digital image. Method 500 is one embodiment of identifying 404 in FIG. 4 above.

Some embodiments of method 500 include identifying 502 a plurality of resources on the network to perform the request on the digital image. Thereafter, method 500 includes presenting 504 the plurality of resources to a user and receiving 506 an indication of a selection from the user, the indicated selection being the identified resource. Method 500 provides decision-making authority to a user as to which resource will perform the request on the image. Other embodiments of identifying 404 the resource are performed by pre-programmed computer instructions and provide no opportunity by human intervention, interaction or override.

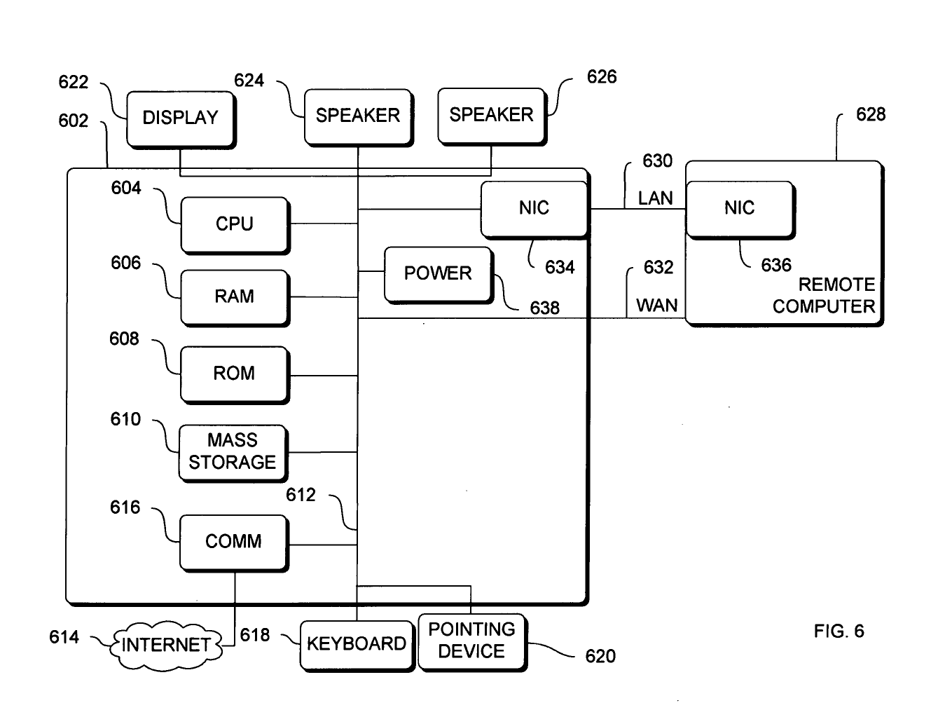

In some embodiments, methods 400-600 are implemented as a computer data signal embodied in a carrier wave, that represents a sequence of instructions which, when executed by a processor, such as processor 604 in FIG. 6, cause the processor to perform the respective method. In other embodiments, methods 400-600 are implemented as a computer-accessible medium having executable instructions capable of directing a processor, such as processor 604 in FIG. 6, to perform the respective method. In varying embodiments, the medium is a magnetic medium, an electronic medium, or an optical medium.

### Hardware and Operating Environment

FIG. 6 is a block diagram of the hardware and operating environment 600 in which different embodiments can be practiced. The description of FIG. 6 provides an overview of computer hardware and a suitable computing environment in conjunction with which some embodiments can be implemented. Embodiments are described in terms of a computer executing computer-executable instructions. However, some embodiments can be implemented entirely in computer hardware in which the computer-executable instructions are implemented in read-only memory. Some embodiments can also be implemented in client/server computing environments where remote devices that perform tasks are linked through a communications network. Program modules can be located in both local and remote memory storage devices in a distributed computing environment.

Computer 602 includes a processor 604, commercially available from Intel, Motorola, Cyrix and others. Computer 602 also includes random-access memory (RAM) 606, read-only memory (ROM) 608, flash memory (not shown) and one or more mass storage devices 610, and a system bus 612, that operatively couples various system components to the processing unit 604. The memory 606, 608, and mass storage devices, 610, are types of computer-accessible media. Mass storage devices 610 are more specifically types of nonvolatile computer-accessible media and can include one or more hard disk drives, floppy disk drives, optical disk drives, and tape cartridge drives. The processor 604 executes computer programs stored on the computer-accessible media.

Computer 602 can be communicatively connected to the Internet 614 via a communication device 616. Internet 614 connectivity is well known within the art. In one embodiment, a communication device 616 is a modem that responds to communication drivers to connect to the Internet via what is known in the art as a “dial-up connection.” In another embodiment, a communication device 616 is an Ethernet® or similar hardware network card connected to a local-area network (LAN) that itself is connected to the Internet via what is known in the art as a “direct connection” (e.g., Ti line, etc.).

A user enters commands and information into the computer 602 through input devices such as a keyboard 618 or a pointing device 620. The keyboard 618 permits entry of textual information into computer 602, as known within the art, and embodiments are not limited to any particular type of keyboard. Pointing device 620 permits the control of the screen pointer provided by a graphical user interface (GUI) of operating systems such as versions of Microsoft Windows®. Embodiments are not limited to any particular pointing device 620. Such pointing devices include mice, touch pads, trackballs, remote controls and point sticks. Other input devices (not shown) can include a microphone, joystick, game pad, satellite dish, scanner, or the like.

In some embodiments, computer 602 is operatively coupled to a display device 622. Display device 622 is connected to the system bus 612. Display device 622 permits the display of information, including computer, video and other information, for viewing by a user of the computer. Embodiments are not limited to any particular display device 622. Such display devices include cathode ray tube (CRT) displays (monitors), as well as flat panel displays such as liquid crystal displays (LCD's). In addition to a monitor, computers typically include other peripheral input/output devices such as printers (not shown). Speakers 624 and 626 provide audio output of signals. Speakers 624 and 626 are also connected to the system bus 612.

Computer 602 also includes an operating system (not shown) that is stored on the computer-accessible media RAM 606, ROM 608, and mass storage device 610, and is and executed by the processor 604. Examples of operating systems include Microsoft Windows®, Apple MacOS®, Linux®, UNIX®. Examples are not limited to any particular operating system, however, and the construction and use of such operating systems are well known within the art.

Embodiments of computer 602 are not limited to any type of computer 602. In varying embodiments, computer 602 comprises a PC-compatible computer, a MacOS®-compatible computer, a Linux®-compatible computer, or a UNIX®-compatible computer. The construction and operation of such computers are well known within the art.

Computer 602 can be operated using at least one operating system to provide a graphical user interface (GUI) including a user-controllable pointer. Computer 602 can have at least one web browser application program executing within at least one operating system, to permit users of computer 602 to access intranet or Internet world-wide-web pages as addressed by Universal Resource Locator (URL) addresses. Examples of browser application programs include Netscape Navigator® and Microsoft Internet Explorer®.

The computer 602 can operate in a networked environment using logical connections to one or more remote computers, such as remote computer 628. These logical connections are achieved by a communication device coupled to, or a part of, the computer 602. Embodiments are not limited to a particular type of communications device. The remote computer 628 can be another computer, a server, a router, a network PC, a client, a peer device or other common network node. The logical connections depicted in FIG. 6 include a local-area network (LAN) 630 and a wide-area network (WAN) 632. Such networking environments are commonplace in offices, enterprise-wide computer networks, intranets and the Internet.

When used in a LAN-networking environment, the computer 602 and remote computer 628 are connected to the local network 630 through network interfaces or adapters 634, which is one type of communications device 616. Remote computer 628 also includes a network device 636. When used in a conventional WAN-networking environment, the computer 602 and remote computer 628 communicate with a WAN 632 through modems (not shown). The modem, which can be internal or external, is connected to the system bus 612. In a networked environment, program modules depicted relative to the computer 602, or portions thereof, can be stored in the remote computer 628.

Computer 602 also includes power supply 638. Each power supply can be a battery.

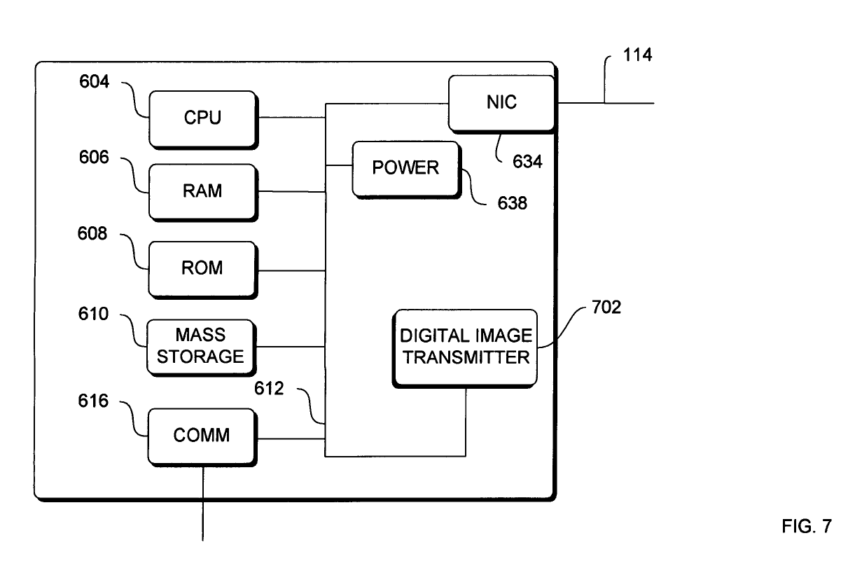

FIG. 7 is a block diagram of the mobile digital medical diagnostic imaging system processor hardware and operating environment 700 to manage digital images.

System 700 is a processor that transmits digital images through network adaptor 634 to a network. System 700 includes a digital image transmitter that accesses a digital image from one of the computer accessible media 606, 608 and/or 610 and transmits the digital image to the network, such as network 114.

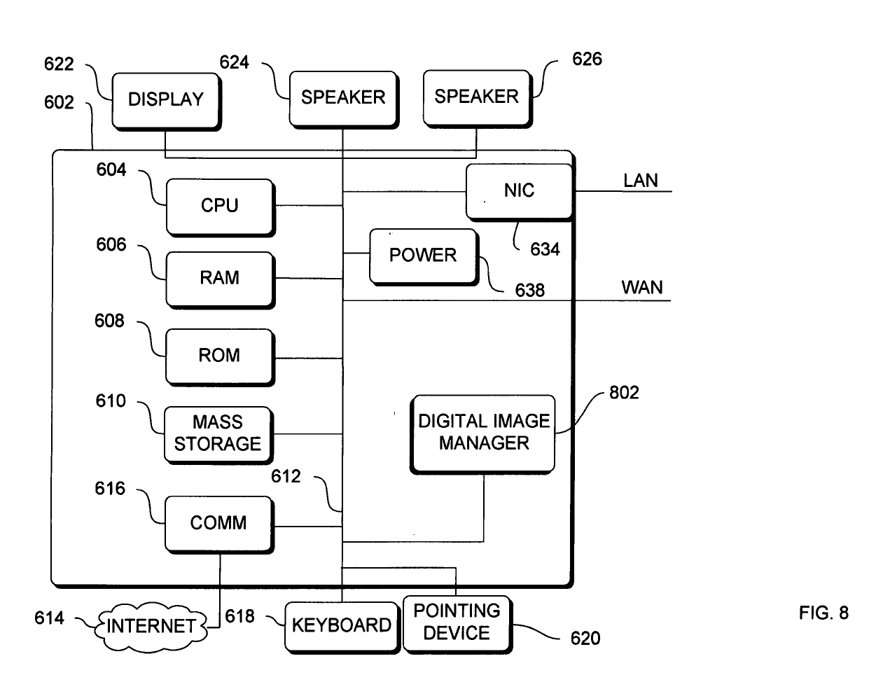

FIG. 8 is a block diagram of a network server hardware and operating environment 800 to manage digital images from a mobile digital medical diagnostic imaging system.

System 800 is a network server, such as network server 112 in FIG. 1 and FIG. 3. The network server 800 performs method 400 and/or method 500. To perform method 400, the network server 800 includes a digital image manager 802. The digital image manager 802 manages digital images from a mobile digital medical diagnostic imaging system on a network.

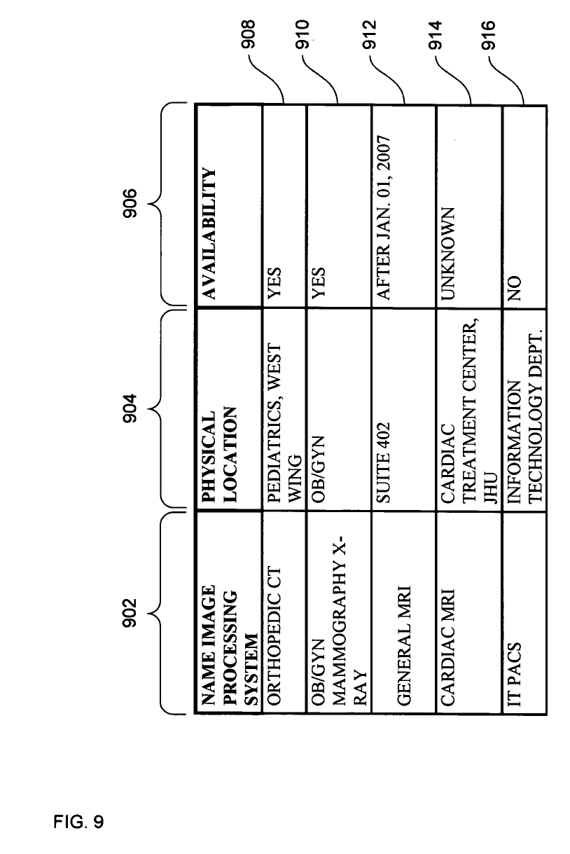

FIG. 9 is a diagram of a layout of a table 900 of a directory for reference in identifying one or more resource on the network to process requests. The directory provides an association between an identity of each resource on the network, the physical location of the resources on the network and an availability of the resource on the network.

Table 900 includes at least 3 columns; a column that describes or represents the name of an image processing system 902, a column that describes a physical location of the image processing system 904, and a column that describes an availability of the image processing system 906. In the example sown in Table 900, 5 rows of exemplary data; row 908, row 910, row 912, row 914 and row 916.

Some embodiments of FIG. 4 and some embodiments of FIG. 5 refer to data in an implementation of Table 900. More specifically, in some embodiments of the action of identifying 404 a resource in method 400 refers to an implementation of table 900 in identifying the name 902 of a resource on the network that is associated with the physical location of the resource 904 on the network and the availability 906 of the resource on the network. In some embodiments of the action in method 500 in FIG. 5 of identifying 502 a plurality of resources on the network to perform the request on the digital image also refers to an implementation of table 900.

### CONCLUSION

A diagnostic imaging network adapted for mobile digital X-Ray imaging processing is described. Although specific embodiments are illustrated and described herein, it will be appreciated by those of ordinary skill in the art that any arrangement which is calculated to achieve the same purpose may be substituted for the specific embodiments shown. This application is intended to cover any adaptations or variations. For example, although described in procedural terms, one of ordinary skill in the art will appreciate that implementations can be made in objected-oriented or any other design methodology that provides the required functions.

In particular, one of skill in the art will readily appreciate that the names of the methods and apparatus are not intended to limit embodiments. Furthermore, additional methods and apparatus can be added to the components, finctions can be rearranged among the components, and new components to correspond to future enhancements and physical devices used in embodiments can be introduced without departing from the scope of embodiments. One of skill in the art will readily recognize that embodiments are applicable to future devices.

The terminology used in this application is meant to include all environments and alternate technologies which provide the same functionality as described herein

We claim:

1. A mobile digital X-Ray imaging system comprising:
a mobile X-Ray unit base;
a column operably coupled to the mobile X-Ray unit base;
a horizontal arm operably coupled to the column;
an X-Ray source operably coupled to the horizontal arm;
a processor operably coupled to the X-Ray source;
a digital X-Ray detector operably coupled to the processor; and
one or more network adapters mounted on the mobile X-Ray unit base and operably coupled to the processor.

2. The system of claim 1, wherein the each of the plurality of network adapters further comprise:
an Ethernet-compliant network.

3. The system of claim 1, wherein the plurality of network adapters further comprises:
two network adapters.

4. A system to process a digital image from a mobile digital medical diagnostic imaging system, the system comprising:
a network;
image processing system operably coupled to the network; and
a network server operably coupled to the network, the network server operable to receive a digital image from the mobile digital medical diagnostic imaging system through the network and operable to transmit the digital image to the image processing system.

5. The system of claim 4, wherein the system further comprises:
a mobile access point operably coupled to the network.

6. The system of claim 4, wherein the network further comprises:
an Ethernet compliant network.

7. The system of claim 4, wherein the image processing system further comprises:
an X-Ray image processing system.

8. The system of claim 4, wherein the image processing system further comprises:
a magnetic resonance imaging system.

9. The system of claim 4, wherein the image processing system further comprises:
a computer tomography imaging system.

10. The system of claim 4, wherein the image processing system further comprises:
a picture archive and communication system.

11. A computer-accessible medium having executable instructions to manage images between a mobile digital medical diagnostic imaging system and a network, the executable instructions capable of directing a processor to perform:
receiving from the mobile digital medical diagnostic imaging system, a digital image and request in reference to the digital image;
identifying a resource on the network to perform the request on the digital image; and
transmitting the request and the digital image to the identified resource.

12. The computer-accessible medium of claim 11, the executable instructions capable of directing a processor to perform the identifying further comprise executable instructions capable of directing a processor to perform:
identifying a resource on the network to perform the request on the digital image in reference to a directory of a plurality of resources on the network, the directory providing an association between an identity of each resource on the network, the physical location of the resources on the network and an availability of the resource on the network.

13. The computer-accessible medium of claim 11, the executable instructions capable of directing a processor to perform the identifying further comprise executable instructions capable of directing a processor to perform:
identifying a plurality of resources on the network to perform the request on the digital image;
presenting the plurality of resources to a user; and
receiving an indication of a selection from the user, the indicated selection being the identified resource.

14. The computer-accessible medium of claim 11, the medium further comprising executable instructions capable of directing the processor to perform:
polling the network for the request.

15. The computer-accessible medium of claim 11, wherein the resource further comprises:
an image processing system.

16. The computer-accessible medium of claim 15, wherein the image processing system further comprises:
an X-Ray image processing system.

17. The computer-accessible medium of claim 16, wherein the image processing system further comprises:
a picture archive and communication system.

18. The computer-accessible medium of claim 11, wherein the request further comprises:
a display request.

19. The computer-accessible medium of claim 11, wherein the request further comprises:
a processing request.

20. A data structure stored on a computer-readable medium to provide reference in identifying one or more resource on the network to process requests, the data structure comprising:
a field storing data representing a name of an image processing system;
a field storing data representing a physical location of the image processing system; and
a field storing data representing an availability of the image processing system.
